(19) United States
(12) Patent Application Publication
(10) Pub. No.: US 2018/0075206 A1
(43) Pub. Date: Mar. 15, 2018

(54) HUMAN CORONARY PASSPORT

(71) Applicant: Ian Lvovich Gabinskii, Sverdlovskaya oblast (RU)

(72) Inventor: Ian Lvovich Gabinskii, Sverdlovskaya oblast (RU)

(21) Appl. No.: 15/475,409

(22) Filed: Mar. 31, 2017

(30) Foreign Application Priority Data
May 5, 2016 (RU) 2016117464

(51) Int. Cl. G06F 19/00; G06K 19/07; A61B 6/03; A61B 6/00

(52) U.S. Cl. CPC G06F 19/3431; A61B 6/503; A61B 6/032; G06K 19/07

(57) ABSTRACT

The invention relates to medicine, therapy, cardiology, in particular it relates to the planning of prevention and treatment of patients in a risk group or already suffering from illnesses such as cardiovascular diseases.

The goal of the proposed invention is the prevention of the risk of developing cardiovascular diseases and an early detection of the risk of fatal cardiovascular complications for all people, especially those not previously assigned to risk groups.

The technical result consists in the development of a universal automated system of monitoring and prevention of the development of cardiovascular diseases, based on a method of identifying the risk of developing cardiovascular diseases.

The technical result is accomplished by virtue of the fact that the present invention represents a universal automated system which is able to identify the risk of developing cardiovascular diseases, plan a treatment, and recommend lifestyle changes, for all people, particularly those not previously assigned to risk groups, with the purpose of warning and preventing CVD (cardiovascular diseases). For the best solution of this problem, the proposed invention of a coronary passport for a person includes: a method of comprehensive evaluation of the coronary status of a person; a system of prevention and monitoring of the risk of development of cardiovascular diseases.

### FIELD OF THE INVENTION

The invention relates to medicine, therapy, cardiology, in particular it relates to the planning of prevention and treatment of patients in a risk group or already suffering from illnesses such as cardiovascular diseases.

### BACKGROUND

Cardiovascular diseases are the foremost causes of mortality and invalidity in the world, especially in view of the gradual trend toward aging of the population of the planet. There is observed a trend toward increasing numbers of people suffering from this pathology. According to data of the World Health Organization, each year 17 million people die from myocardial infarct and other illnesses of the cardiovascular system.

It is well known that the risk of cardiovascular diseases increases with age, but in the modern world the risk zone also rather often includes quite young people. The lifestyle of the majority of modern-day people leaves much to be desired: low physical activity, improper nutrition, stressful situations, bad habits. As a result, diseases of the cardiovascular system are rapidly “growing younger”. Obesity, smoking, improper nutrition, metabolic disorders, physical and psycho-emotional overload are the main causes in the development of heart disease.

Unlike other diseases, the development of cardiovascular diseases may be prevented in many cases with the help of simple measures for lifestyle change. At present it is widely accepted that everyone should strive for a healthy lifestyle in order to prevent cardiovascular diseases, but there are groups of people who have a higher risk of occurrence of cardiovascular diseases, such as infarct or stroke. Thus, it is desirable to effectively detect persons who are vulnerable to cardiovascular diseases, and to prescribe prevention or treatment programs for them which, including programs directed at changing lifestyle, in order to improve the person's state of health.

### SUMMARY OF THE INVENTION

The principal problem at present is the existence of many cases of asymptomatic development of ischemic heart disease, when the first manifestation of the disease is an acute myocardial infarction or sudden coronary death. It is important to point out that this group of people, who are generally considered healthy because they show no symptoms of any kind, need to be assigned to the elevated risk group. The method of monitoring their cardiovascular health should be easy, not time-consuming and not expensive. Moreover, since they feel themselves to be healthy, an important task is motivating them to undergo diagnosis and treatment or change their lifestyle. The results of the coronary health status should be presented in a way which allows people to understand the need to follow the doctor's recommendations.

In order to develop a system for monitoring of the cardiovascular health status of a patient, it is first of all necessary to determine the risk of possible development of cardiovascular diseases. The patent and scientific literature describes methods for determining the risk of developing cardiovascular diseases on the basis of several parameters.

One of the existing methods of determining risks is the Framingham scale, which is designed to calculate the risk of coronary death and an overall indicator of fatal and nonfatal coronary events in the next 10 years for people with HDL cholesterol of 1.3-1.53 mmol/l and not taking drugs to lower the arterial pressure (according to the NCEP ATP 3 of 2002) [Wilson P. W., D'Agostino R. B., Levy D., et al. Prediction of coronary heart disease using risk factor categories//Circulation.-1998.-No 12.-Vol. 97.-p. 1837-1847; Brindle P., Emberson J., Lampe F. et al. Predictive accuracy of the Framingham coronary risk score in British men: prospective cohort study//BMJ.-2003.-Vol. 325.-p. 1267-1270.]. This scale cannot be used for people with other levels of HDL cholesterol, or when evaluating risk in a setting of taking drugs to lower the arterial pressure. Furthermore, the study was done for an American population, and when used in other regions there is an exaggeration of the real absolute risk [Brindle P., Fahey T. Primary prevention of coronary heart disease//BMJ.-Vol. 325.-p. 56-57].

The next primary known method for identifying risks of developing cardiovascular diseases is the European scale for evaluating individual cardiovascular risk, or SCORE (Systematic Coronary Risk Evaluation), which considers gender, age, smoking and education status, and total cholesterol concentration in the blood [Conroy R. M., Pyorala K., Fitzgerald A. P., et al. SCORE project group. Estimation of ten-year risk of fatal cardiovascular disease in Europe: the SCORE project//Eur. Heart J.-2003.-Vol. 24 (11).-p. 987-1003]. The developers of the SCORE scale pointed out that the evaluation of overall risk by this scale should be adapted to the individual European populations depending on national conditions, resources and priorities. There is a variant of the scale adapted to the Russian population [Shal'nova S. A., Oganov R. G., Deyev A. D. Evaluation and control of risk of cardiovascular diseases for the population of Russia//Kardiovaskulyarnaya terapiya i profilaktika-2004.-No 4.-p. 4-11], the distinguishing feature of which is the inclusion among the prognostic factors of a discriminator specific to the Russian population—the education level—and also a number of other factors (relative body mass, heart rate, level of high density lipoprotein cholesterol). The Russian scale, like the European, predicts fatal events, the threshold for high risk being defined as more than 5% in ten years. The SCORE scale, both European and Russian, has limits in its use, since it can only be applied to persons with no clinical signs of atherosclerotic illnesses, which significantly restricts the use of the scale in actual practice. Patients with an established diagnosis of cardiovascular disease, with sugar diabetes, and/or with a total cholesterol level over 8.0 mmol/l or AP greater than 180/110 mm are assigned to the high risk category, and the calculation of the overall risk by SCORE cannot be used for them. Whereas in certain patients with clinical signs of atherosclerosis the disease has a stable course for many years and does not end with any serious complications.

A drawback of the known methods of determining risk is that the prognostication of individual risk does not ensure an absolute accuracy of the result. The accuracy of a determination of the risk of developing cardiovascular complications by these methods does not exceed 50% [Chobanian A. V., Bakris G. L., Black H. R. et al. Joint National Committee on Prevention, Detection, Evaluation, and Treatment of High Blood Pressure, National Heart, Lung, and Blood Institute, National High Blood Pressure Education Program Coordinating Committee: Seventh report of the Joint National Committee on Prevention, Detection, Evaluation, and Treatment of High Blood Pressure.//Hypertension.-2003.-Vol. 42.-p. 1206-1252].

Evaluation of the risk of development cardiovascular diseases by the known methods is calculated by the doctor with the use of a program or table. Calculation of risks is used to determine the category of the risk of developing a cardiovascular disease by a person: low, medium or high. On the basis of the risk category as determined by one of the known methods, it is possible to recommend a treatment in keeping with worldwide medical recommendations. The generating of such recommendations for the existing risk groups is disclosed in U.S. Pat. No. 4,464,122. This system of generating a summary in the form of commentaries and recommendations in regard to the health of a patient is designed to call attention not only to the physical condition of the subject, but only aimed at improving the state of health by using proposed changes in lifestyle.

The closest to the proposed solution is U.S. Pat. No. 7,647,234, disclosing a system which performs a diagnostics and prescribes treatments for cardiovascular diseases. Data is entered from the patient's chart and, with the use of an automated system, the patient is recommended measures for prevention and treatment. This solution does not give consideration to patients who clearly cannot be assigned to the risk group for developing cardiovascular diseases.

A study of the scientific and patent literature has not found the combination of significant indicators as disclosed in the invention.

The goal of the proposed invention is the prevention of the risk of developing cardiovascular diseases and an early detection of the risk of fatal cardiovascular complications for all people, especially those not previously assigned to risk groups.

The technical result consists in the development of a universal automated system of monitoring and prevention of the development of cardiovascular diseases, based on a method of identifying the risk of developing cardiovascular diseases.

On the whole, the terms used in the specification should be interpreted as they are known to the specialist in the given field of technology. Certain terms are defined below, in order to provide additional clarity. In the case of a conflict between the known meaning and the presented definition, the presented definition should be used.

By the term “coronary code” is meant the classification of patients which is based on the combination of a visualization of the patient's coronary arteries with the overall cardiovascular risk as determined by a specially developed method.

By the term “coronary status” is meant the classification of groups of patients based on the patient's coronary code.

By the term “coronary group” is meant the classification of groups of patients based on the patient's coronary code GABICA (Groups of Arterial Blockage Interpreted as Coronary Aggravation), including the degree of risk of fatal coronary complications, and a program of therapeutic and preventive measures reducing the risk of future fatal complications in the next 2-3 years.

The term “coronary passport” means a new modern medical technical principle for increasing the efficacy of therapeutic and preventive measures, by preserving the patient's coronary health and improving the patient's quality of life, performed on the basis of modern non-invasive and minor invasive methods of coronary vessel diagnostics and determination of the overall cardiovascular risk by a specially developed method providing new approaches to doctor/patient interaction, the formation of monitoring programs for coronary status on the basis of the GABICA classification, and access to global specialized information resources.

The technical result is accomplished by virtue of the fact that the present invention represents a universal automated system which is able to identify the risk of developing cardiovascular diseases, plan a treatment, and recommend lifestyle changes, for all people, including those not previously assigned to risk groups, with the purpose of warning and preventing CVD (cardiovascular diseases). For the best solution of this problem, the proposed invention of a coronary passport for a person includes: a method of comprehensive evaluation of the coronary status of a person; a system of prevention and monitoring of the risk of development of cardiovascular diseases.

The method of comprehensive evaluation of the coronary status of a person includes a determination of the coronary code with the use of the technologies of computer tomography, coronaro-angiography, the SCORE table, and the GABICA table of risk factors for cardiovascular diseases, which is used to determine on the scale of coronary risks whether the coronary code belongs to one of the coronary groups. The method of comprehensive evaluation of the coronary status of a person is carried out with the aid of the hardware and software complex for the examination and the medical history.

The system of prevention and monitoring of the risk of development of cardiovascular diseases includes the following basic processes: scheduling of an appointment, determination of the coronary status, conducting of therapeutic and preventive measures, follow-up systematic examination of the coronary status, the processes being performed with the use of a specialized instrument for nosological identification of a person having for example the shape of an electronic plastic card, which is the key of the automated system of doctor/patient interaction, making it possible to use Internet technologies to obtain the results of the examination, technologies of therapeutic and preventive measures, and programs of prevention and treatment.

### BRIEF DESCRIPTION OF THE FIGURES

The variant embodiment of the present invention presented below will be described only as an example with references to the enclosed drawings, in which:

FIG. 1—external appearance of the instrument for identification of a person, having the form of an electronic plastic flash card.

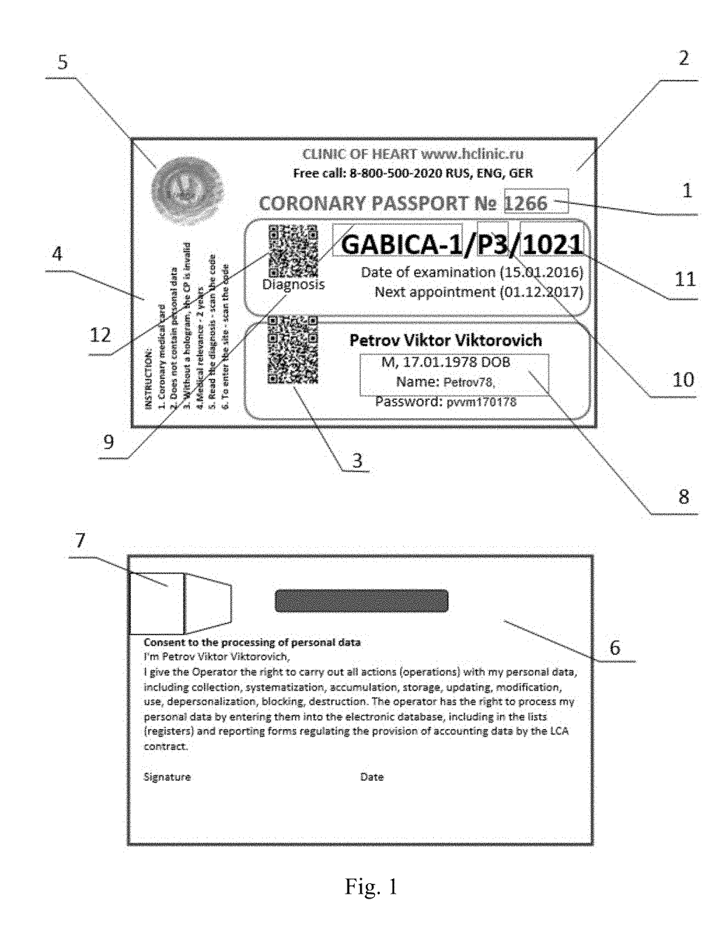

FIG. 2—basic principles for obtaining the coronary code, the coronary status and the coronary group are presented

### DETAILED DESCRIPTION OF THE INVENTION

The following detailed description of the invention is presented for a more clear illustration thereof. However, it will be understood by the specialists that the inventive concept of the present invention is not limited by these specific details.

In one variant embodiment of the invention, a coronary passport for a person is in the form of an electronic plastic flash card which contains the following information:

Name and number of the document 1, such as CORONARY PASSPORT No 1266.

Contact data for the clinic 2, including telephone number and web site address of the clinic.

Reference to the clinic's site 3 for login to the personal account, presented for example in QR code format.

Mini instruction 4 on the use of the coronary passport, containing a minimal set of rules, such as:
The coronary passport does not work without a hologram.
Medically good for 2 years.
Code must be scanned to read the diagnosis.
Code must be scanned for login to the personal account of the site.

A hologram 5 confirming the authenticity of the document.

Consent to the processing of personal data 6.

Memory card 7, containing for example an electronic card of the patient with all of their analysis results.

Data of the patient's account record for login to the clinic site.

Primary clinical diagnosis of the patient 8.

Coronary group of the person 9, including their treatment tactics, such as GABICA-1.

Therapeutic and preventive program 10.

Coronary status of the patient 11.

Date of examination and date of next appointment 12.

The coronary status carries information about the condition of the coronary arteries of the patient, the overall cardiovascular risk, and the presence of PCI and/or CABG interventions, which is necessary for the prevention, diagnostics and monitoring of coronary health. According to the coronary status, 5 GABICA coronary risk groups have been identified, on the basis of which patient monitoring and therapeutic and preventive measures are carried out according to 10 programs.

One of the variants of the coronary status can be a code consisting of four numerals, where:

The first numeral indicates the result of the visualization of the coronary arteries according to computer tomography [CT] data.

The second numeral is the result of the visualization of the coronary arteries by the coronaro-angiography [CAG] method.

The third numeral corresponds to the absolute risk of fatal cardiovascular complications as determined by the specially developed scale SE.

The fourth numeral reflects the presence of PCI and/or CABG interventions in the medical history.

An interpretation of the values of the digits in the coronary code is presented in table 1.

TABLE 1

| Position | Value | Rule |
|---|---|---|
| 1st digit indicates | 0 | None performed or not possible to obtain |
| generalized result | | reliable information on the condition of |
| of MS CT-CAG | | the coronary arteries by the method of |
| tests | | CT-CAG (due to rhythm disorder, due to |
| | | pronounced calcification) |
| | 1 | CT-CAG with no signs of atherosclerotic |
| | | changes to the coronary arteries (primary |
| | | prevention) |
| | 2 | Incipient changes to the coronary arteries |
| | | with no significant stenosis (LCA trunk |
| | | less than 30%, other CA less than 50%) |
| | | (conservative treatment tactic, dynamic |
| | | observation, if need be repeat CT-CAG |
| | | every 6 months) |
| | 3 | CT-CAG shows stenosis requiring invasive |
| | | angiography examination (borderline |
| | | stenosis 50-69%, lesions in main trunk |
| | | 30-49%) (regularly scheduled CAG) |
| | 4 | CT-CAG data shows significant stenosis |
| | | and occlusion of the coronary arteries. |
| | | Ostium lesions, RCA lesions, LCA |
| | | lesions (emergency CAG) |
| 2nd digit indicates | 0 | No CAG performed |
| generalized result | 1 | No changes in the coronary arteries |
| of CAG tests | | according to CAG results |
| | 2 | CAG shows changes in the coronary arteries |
| | | not requiring invasive intervention (stenting) |
| | 3 | Based on CAG data patient requires |
| | | percutaneous intervention (stenting, |
| | | angioplasty) |
| | 4 | CAG results show that patient requires |
| | | surgical treatment (CABG) |
| 3rd digit, scale SE | 0 | =Score + GABICA |
| (SE = SCORE + | 1 | =Score + GABICA |
| GABICA) | 2 | =Score + GABICA |
| | 3 | =Score |
| | 4 | =Score |
| | 5 | =Score |
| | 6 | =Score |
| 4th digit, presence | 0 | No interventions |
| of PCI and/or | 1 | Stents were installed |
| CABG interventions | 2 | CABG has been performed |
| in patient's | 3 | Stents were installed and CABG performed |
| medical history | | |

The absolute risk of fatal cardiovascular complications as determined by the specially developed SE scale, which evaluates the overall risk according to the SCORE and GABICA scale.

The SCORE scale is designed to calculate fatal cardiovascular complications. Fatal cardiovascular complications (events) include: death from myocardial infarct, other forms of ischemic heart disease, stroke, including sudden death and death within 24 hours of appearance of symptoms, death from other noncoronarogenic cardiovascular diseases with the exception of definite non-atherosclerotic causes of death. The SCORE scale is not used in patients with proven cardiovascular diseases of atherosclerotic genesis (CHD, cerebrovascular diseases, aortal aneurism, atherosclerosis of the peripheral arteries), sugar diabetes type I and II affecting the target organs, chronic kidney diseases, in persons with very high levels of individual risk factors, citizens over the age of 65 years (these groups have the highest level of overall 10-year cardiovascular risk) and citizens under the age of 40 years, since regardless of presence of risk factors (with the exception of very high levels of individual factors) they have a low absolute risk of fatal cardiovascular complications in the next 10 years of their lives. The scale has been developed on the basis of the following factors: age, gender, smoking, level of systolic AP and TC.

According to the classification of risks by the SCORE scale, all patients are divided into 4 groups:

1—low risk, less than 1%

2—medium risk, from >1 to 5%

3—high, from >5% to 10%

4—very high, >10%

The SCORE scale is designed for patients aged 40 years up to 60 years.

Automatically placed in the very high risk category according to recommendations (regardless of the SCORE scale) are patients having cardiovascular diseases of atherosclerotic genesis (ischemic heart disease, cerebrovascular disease: ischemic stroke, brain hemorrhaging, transitory ischemic attack, aortal aneurism, clinically pronounced atherosclerosis of the peripheral arteries); patients with diabetes type 1 and 2, affecting target organs (microalbuminuria); patients with CRF and symptoms of kidney failure of moderate to severe degree (glomerular filtration rate (GFR) <60 ml/min/1.73 M2); patients with arterial hypertension of 3rd degree (AP ≧180/110 mm); patients with family history of high cholesterol; those aged >65 years; patients with total cholesterol figures >8 mmol/l; those with heart failure, including heart failure with intact ejection fraction stage II by the Strazhesko-Vasilenko classification.

Not being part of the code, but considered in the 3rd digit in the unified SE scale, is the GABICA scale (extra factors increasing risk), reflecting the cardiovascular risk in dependence on influencing factors of risk not included in the SCORE scale. When the SCORE scale is used, the risk of cardiovascular diseases is likely underestimated, since no consideration is given to important factors for the risk of atherosclerosis. It is therefore necessary to identify the cardiovascular risk in dependence on factors of asymptomatic impairment of the organs. Evaluation of the overall cardiovascular risk is of crucial importance in selecting a preventive strategy for persons who generally have a combination of several risk factors. The risk factors mutually potentiate each other. Even at low levels of individual risk factors, the overall risk may be significant. This is especially important in young patients (≧30 to 40 years) who have not been included in the SCORE scale, as well as patients with low risk on the SCORE scale.

All the patients are divided into 2 groups, low risk and high risk

1—the low risk group is patients scoring <5 points

2—the high risk group is patients scoring ≧5 points (5 or more points).

If the risk is defined as low (<1%) or medium (≧1% <5%) by the SCORE scale, an evaluation is made for the following indicators and a risk category is assigned depending on the points scored. The scale of risk categories by this classification runs from 0 to 8 points (min-0, max-9).

The indicators which are evaluated in the evaluation of the cardiovascular risk in dependence on risk factors and asymptomatic impairment of the organs are:

fasting plasma glucose 5.6 to 6.9 mmol/l—1 point;

rise in C-reactive protein (>5 mg/l)—1 point;

body mass index >30 kg/M2—0.5 points;

abdominal adiposity (waist circumference >100 cm)—1 point;

HDL <1.0 for men and <1.2 for women—1 point;

subclinical lesions of carotid arteries (stenosis >50%)—0.5 points;

hereditary history of early cardiovascular diseases in relatives of the first degree aged <45 years for men and <55 years for women—1 point;

lack of exercise—0.5 points;

hypertrophy of the left ventricle—0.5 points;

occupation involving heightened psycho-emotional stress (drivers, pilots, firemen, train engineers, reporters, etc.)—1 point;

family situation (bachelors)—0.5 points;

depression—0.5 points. Depression can be evaluated by the Beck scale, for example.

The obtained coronary codes are then used to form the coronary status of the patient, on the basis of which the patients are combined into special coronary groups according to the degree of coronary risks (see tables 2, 3) for the prescribing and carrying out of therapeutic and preventive measures (coronary treatment programs, table 4), substantially lowering the risk of future fatal coronary complications.

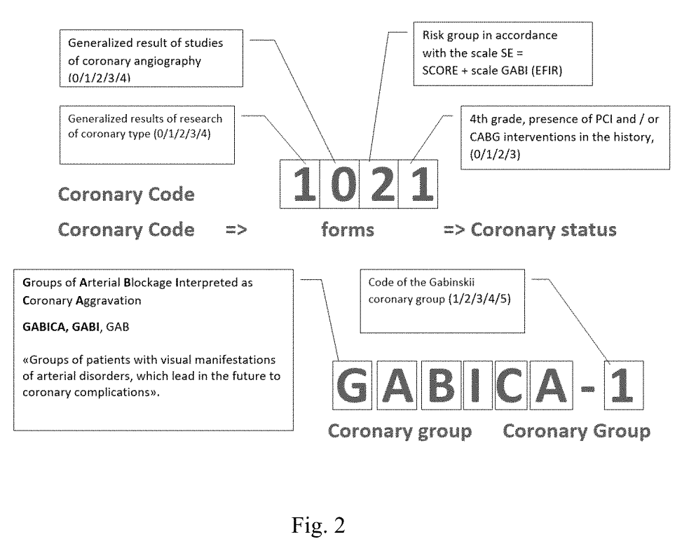

The principles of obtaining the coronary code, the coronary status, and the coronary group are presented in FIG. 2.

The automated system of doctor/patient interaction to determine the coronary status includes:
1. Scheduling an appointment and booking an appointment time, using an Internet portal, online registration, and the personal account of the patient, making use of global services for access to the appointments journal;
2. Scheduling of services, specifically financial-legal registration, confirmation of appointment time;
3. Patient examination and filling out forms, on the basis of which the patient groups are formed: general technological and special;
4. Investigation of visualization of coronary arteries per MSCT AG CA data, and CAG, after which an interaction of the CT system and the coronaro-angiography system with the MIS MEDOFIS occurs, by performing an analysis in a special program, transmittal of initial data to the MEDOFIS database, filling out a form;
5. Laboratory tests, test results automatically to into the patient's medical chart;
6. Final conclusion as to coronary status, on the basis of which the coronary group is formed for prescribing and performance of therapeutic and preventive measures (coronary treatment programs), significantly lowering the risk of future fatal coronary complications. The type of program and its scope are determined (monitoring, prevention, therapy or rehabilitation). Prescriptions and recommendations are made based on the results.

It should be noted that the aforementioned variants illustrate, but do not limit the invention.

1. Method of comprehensive evaluation of the coronary status of a person includes a determination of the coronary code with the use of the technologies of computer tomography, coronaro-angiography, the SCORE table, and the GABICA table of risk factors for cardiovascular diseases, which is used to determine on the scale of coronary risks whether the coronary code belongs to one of the coronary groups.

2. Method of comprehensive evaluation of the coronary status of a person according to claim 1, characterized in that it is carried out with the aid of the hardware and software complex for the examination and the medical history.

3. System of prevention and monitoring of the risk of development of cardiovascular diseases includes the following basic processes: scheduling of an appointment, determination of the coronary status, conducting of therapeutic and preventive measures, systematic examination of the coronary status, the processes being performed with the use of a specialized instrument for nosological identification of a person, which is the key of the automated system of doctor/patient interaction, making it possible to use Internet technologies to login to the personal account of the patient.

4. System of prevention and monitoring of the risk of development of cardiovascular diseases according to claim 3, characterized in that the instrument for nosological identification of a person is in the form of an electronic plastic card, which is the key of the automated system of doctor/patient interaction.

5. System of prevention and monitoring of the risk of development of cardiovascular diseases according to claim 4, characterized in that the system of doctor/patient interaction makes it possible to use Internet technologies for:
a. Scheduling an appointment and booking an appointment time, using an Internet portal, online registration, and the personal account of the patient, making use of global services for access to the appointments journal;
b. Scheduling of services by the patient, specifically financial-legal registration, confirmation of appointment time;
c. Patient examination by the doctor and filling out forms, on the basis of which the patient groups are formed: general technological and special;
d. Entering by doctor of results of investigation of visualization of coronary arteries per MSCT AG CA and CAG data, after which an interaction of the CT system and the coronaro-angiography system with the MIS MEDOFIS occurs, by performing an analysis in a special program, transmittal of initial data to the MEDOFIS database, filling out a form;
e. Automatic filling out of patient's medical chart;
f. Final conclusion as to coronary status, on the basis of which the coronary group is formed for prescribing and performance of therapeutic and preventive measures (coronary treatment programs), significantly lowering the risk of future fatal coronary complications. The type of program and its scope are determined (monitoring, prevention, therapy or rehabilitation).
